(19) United States
(12) Plant Patent Application Publication
(10) Pub. No.: US 2003/0172428 P1
(43) Pub. Date: Sep. 11, 2003

(54) ZOYSIAGRASS PLANT NAMED 'SERENE'

(76) Inventor: Ronald Douglas Nixon, Midway, AL (US)

Correspondence Address: Ronald Nixon, 2405 Hwy 82, Midway, AL, 36053, US

(21) Appl. No.: 10/093,810

(22) Filed: Mar. 11, 2002

(51) Int. Cl. A01H 5/00

(52) U.S. Cl. PLT/390

(57) ABSTRACT

An asexually reproduced variety of perennial zoysiagrass with a unique combination of characters including fine leaf blade and low floral production.

### BRIEF SUMMARY OF THE INVENTION

### Background of the Invention

This invention relates to a new and distinct perennial zoysiagrass cultivar identified as ‘Serene’ zoysiagrass (herein referred to as ‘Serene’). I discovered ‘Serene’ under cultivated conditions in a sod field near Midway, Ala. ‘Serene’ was identified as a distinctly different vegetative patch or segregated clonal plant differing by finer leaf texture from the variety ‘Meyer’. The grass identified as ‘Serene’ differed from ‘Meyer’ in that after sod harvest, ‘Serene’ continued growth with a finer leaf texture. I asexually reproduced ‘Serene’ by taking vegetative cuttings of the plant material from the field including rhizomes and tillers as sod, cutting the rhizomes and sod into segments, each with a vegetative bud, and rooted them in field soil and potting media near Midway, Ala.

For purposes of registration under the “International Convention for the Protection of New Varieties of Plants” (generally known by its French acronym as the UPOV Convention) and noting Section 1612 of the Manual of Plant Examining Procedure, it is proposed that the title of the invention is Zoysiagrass plant named ‘Serene’.

### BRIEF DESCRIPTIONS OF THE ILLUSTRATIONS

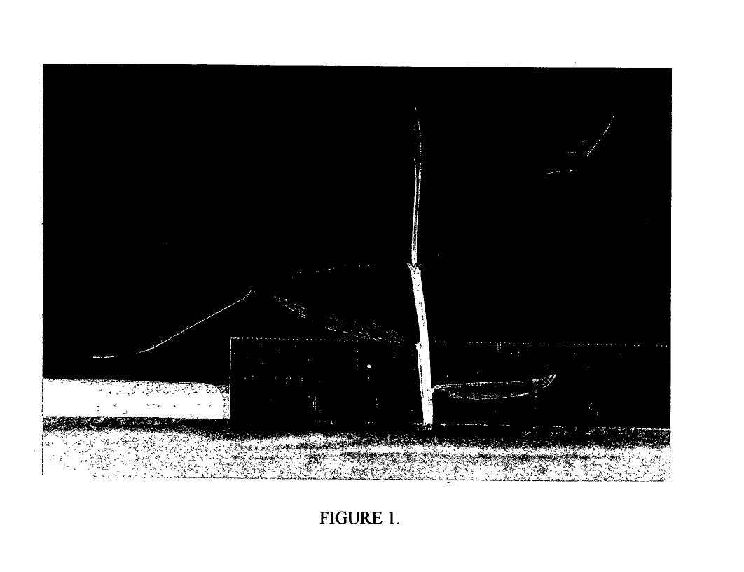

FIG. 1. Tiller of ‘Serene’ zoysiagrass.

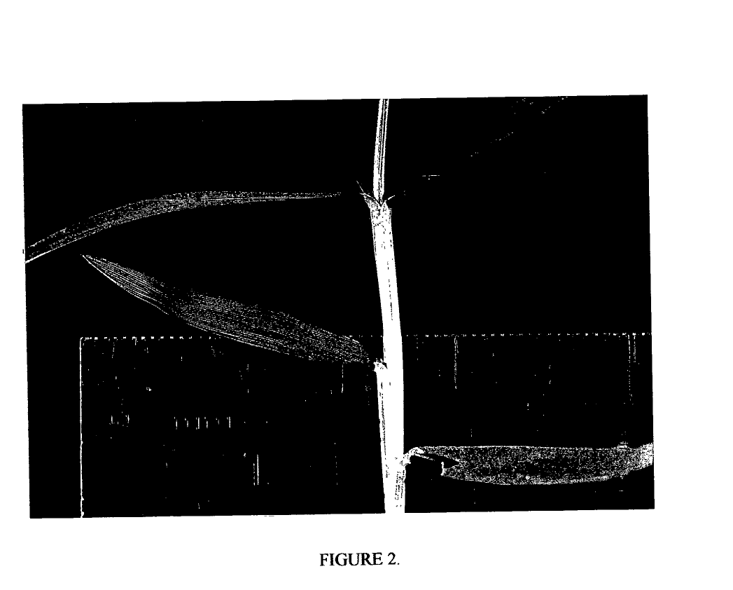

FIG. 2. Leaf surface of ‘Serene’ zoysiagrass.

### COMPLETE DESCRIPTION OF THE VARIETY

‘Serene’ was characterized in greenhouse and field conditions. ‘Serene’ is a unique variety of zoysiagrass ((Zoysia japonica (L.)) Merr. that was discovered under cultivated conditions as finer in leaf texture and rate of re-growth after harvest than ‘Meyer’. ‘Serene’ was propagated by me under field and greenhouse conditions by cutting of rhizomes and tillers, rooting them in soil, and planting of the rooted material to provide planting stock for studying performance and for comparison of morphological characters after propagation. ‘Serene’ has been propagated by rhizomes, tiller, and sod. No seedling establishment from ‘Serene’ has been noticed in either greenhouse or field studies.

‘Serene’ spreads primarily by rhizomes. Characteristics of ‘Serene’ as follows were taken at time of first flowering in winter with a 14-hour daylength from greenhouse grown plants. The greenhouse was located near Lebanon, Ore., with a nighttime low of 50 degrees F., and a daytime high of 80 degrees F., and a minimum soil temperature of 77 degrees F. The plants were held under natural daylength, supplemented with photosynthetically active radiation equivalent to approximately 50% sunlight. The plants were fertilized with the equivalent of 1 pound of actual N per month, using a soluble fertilizer of 20-20-20 in 2 equal soluble applications per month.

The rhizomes of ‘Serene’ have a mean internode length of 22.4 mm as measured between the second and third nodes or third and fourth nodes, with a mean rhizome node dimension 1.8 mm near and above the second and third youngest nodes. The rhizomes of ‘Serene’ root adventitiously at the nodes.

The leaf blades of ‘Serene’ are rolled in the bud, and are flat and stiff. The upper leaf surface has no hairs on the upper or lower leaf surface, similar to ‘Diamond’ (U.S. Plant Pat. No. 10,636). The third youngest vegetative leaf blade has a mean of 28.5 mm in length and a mean width of 1.8 mm. Serene has a finer leaf blade width than ‘Meyer’ (unpatented) (Table 1). Leaf sheath length of the third youngest leaf had a mean value of 18 mm in length. Color notations of plant tissues were based on the Munsell Color Charts for Plant Tissues, Munsell Color, Baltimore, Md., 1977. Light quality, photoperiod, and general growth of the plants affect color notations.

Measured under greenhouse conditions in February 2002, the genetic, adxial leaf color of ‘Serene’ is 5GY ⅝. The ligule of ‘Serene’ has 5-7 hairs up to 1.5 mm long in the center of ligule. The inflorescence of ‘Serene’ is a terminal spike-like raceme, with spikelets on short pedicels. ‘Serene’ shows a lower propensity for floral production (Table 2) from Cashmere (unpatented) and Emerald (unpatented).

TABLE 1
Mean of four leaf blade width of zoysiagrass varieties from
greenhouse grown plants, Lebanon, OR, February 2002.

| | 3^{rd} Youngest Leaf blade width |
|---|---|
| Variety | mm |
| Serene | 1.80 |
| Diamond | 1.54 |
| Meyer | 3.40 |
| Crowne | 3.04 |
| El Toro | 3.70 |
| Palisades | 3.64 |
| Emerald | 1.84 |

TABLE 2
Floral production of zoysiagrass varieties from
greenhouse grown plants, Lebanon, OR, November 2001.

| | Flowering | |
|---|---|---|
| | Frequency | Mean Raceme |
| Variety | Number | Number |
| Serene | 0 | 0.0 |
| Cashmere | 100 | 10.3 |
| Meyer | 0 | 0.0 |
| Emerald | 100 | 16.3 |

### References Cited

U.S. Patent Documents

| | | |
|---|---|---|
| October, 1998. | Engelke, M.C. | U.S. PP 10,636. ‘Diamond’ |
| | | zoysiagrass |
| October, 2000. | Engelke, M.C. | U.S. PP 11,570. ‘Crowne’ |
| | | zoysiagrass |
| December, 1986. | Youngner, V. B. | U.S. PP 5,845. ‘El Toro’ |
| | | zoysiagrass |
| September, 2000. | Engelke, M.C. | U.S. PP 11,515. ‘Palisades’ |
| | | zoysiagrass |

I claim:

1. A new and distinct variety of zoysiagrass plant, substantially as described and illustrated herein, characterized particularly by having principle distinguishing characteristics of fine leaf blade and low floral production.
